(19) United States
(12) Patent Application Publication
(10) Pub. No.: US 2014/0046409 A1
(43) Pub. Date: Feb. 13, 2014

(54) LIGHT SOURCE APPARATUS FOR PHOTO-DIAGNOSIS AND PHOTOTHERAPY

(71) Applicant: KOREA ELECTROTECHNOLOGY RESEARCH INSTITUTE, Changwon-si (KR)

(72) Inventors: Uk KANG, Seoul (KR); Dae Sic LEE, Seoul (KR); Guang Hoon KIM, Busan (KR); Min Woong JUNG, Seoul (KR); Garry V. PAPAYAN, ST. PETERSBURG (RU); Vladimir BEREZIN, Ansan-si (KR)

(73) Assignee: KOREA ELECTROTECHNOLOGY RESEARCH INSTITUTE, Changwon-si (KR)

(21) Appl. No.: 13/853,466

(22) Filed: Mar. 29, 2013

(30) Foreign Application Priority Data
Aug. 9, 2012 (KR) 10-2012-0087175

(51) Int. Cl. F21V 9/08; A61N 5/06

(52) U.S. Cl. CPC F21V 9/08; A61N 5/06
USPC 607/89; 607/88

(57) ABSTRACT

Disclosed herein is a light source apparatus for photo-diagnosis and phototherapy. The light source apparatus includes a first light source, a second light source, a light-guide, an interference filter, and a compensation filter. The first light source is non-coherent, and the second light source is coherent. The light-guide delivers light emitted from the first light source and the second light source. The interference filter is disposed on an optical path of the first light source. The compensation filter is disposed between the first light source and the light-guide, and compensates for an output spectrum of the first light source and converts the output spectrum of the first light source into a predetermined reference output spectrum. Here, the light emitted from the second light source is reflected by the interference filter to be incident to the light-guide and the light from the first light source passes through the interference filter at the same time.

### CROSS-REFERENCE TO RELATED APPLICATION

This application claims under 35 U.S.C. §119(a) the benefit of Korean Patent Application No. 10-2012-0087175 filed Aug. 9, 2012, the entire contents of which are incorporated herein by reference.

### BACKGROUND

(a) Technical Field

The present invention relates to a light source apparatus. More particularly, the present invention relates to a light source apparatus for photo-diagnosis and phototherapy, which is configured to effectively irradiate light through a light-guide to increase the accuracy of the photo-diagnosis and the efficiency of the phototherapy with respect to diseases occurring in the inner and outer parts of the body, particularly, tumors including cervical cancer.

(b) Background Art

Today, phototherapy to treat skin diseases, including acne, freckle, age spots, blemish, scar, wrinkle and malignant tumor, is well-known. The phototherapy devices used for such medical-purpose phototherapy usually include the source of treatment ray and the optical cable formed of optical fiber that delivers light generated from a source to the treatment parts of patients.

Various kinds of lamps using halogen, xenon, metal-halide, mercury and other materials are being used as the source, and an optical fiber light source apparatus based on such lamps is disclosed in the U.S. Pat. No. 6,461,866.

U.S. Pat. No. 5,634,711 discloses a light source using an LED array, and U.S. Pat. No. 7,016,718 discloses a light source apparatus using a coherent laser light source.

On the other hand, as an example of existing light source for phototherapy, the light source from Lumacare Inc. that was developed to perform the Photo Dynamic Therapy (PDT) includes only a halogen lamp.

Such exclusive use of halogen lamp does not provide a sufficient intensity of light within an acceptable range in case of a treatment in which the spectrum light in the short-wavelength range under 400 nm is used. Also, when a single lamp is used, it is difficult to form the optimal condition that satisfies the various requirements for the diagnosis and treatment.

The light sources are selected by production requirements for the apparatus in consideration of the technological and economic aspects and means of special medical purposes. Particularly, when a complex operation is necessary, the use of a single lamp cannot provide an optimal method. In this case, the apparatus developer relies on the lamp that has a special function, or simultaneously uses a plurality of lamps to supplement the shortcomings.

There are some known methods that allow a user to use a plurality of light sources as needed in order to supplement the optical output power or wavelength given by a single light source.

For example of methods for replacing light sources, appropriate light sources can be coaxially arranged at the end side of a light guide cable by a rotation method without a change of a distance between a light-guide cable and a light source, or light sources can be moved in a longitudinal axis direction by a motor as disclosed in U.S. Pat. No. 6,494,899.

Alternatively, the lamp is fixed, and light can be sequentially incident to the incidence plane of the light-guide by a movable folding-type mirror.

However, this lighting method has limitations in that (a) the apparatus becomes complicated due to the moving light sources or mirror and (b) light irradiated from a plurality of light sources cannot be simultaneously used.

On the other hand, lights with two or more different wavelengths are needed to be irradiated to an object of measurement in order to efficiently perform fluorescence diagnosis and photo dynamic therapy.

For such irradiation of light, a combinational use of lamp and laser can be considered. For example, a mercury lamp that irradiates light with a wavelength range of 350 nm to 450 nm and a laser that has a single wavelength of 635 nm can be used for the fluorescence diagnosis without using a fluorescent contrast medium.

While the mercury lamp provides a background image that provides information on the shape of a tissue by simultaneously exciting endogenous fluorescent materials (collagen, keratin, NADH, and FAD) that widely and evenly exist in the skin, the laser allows a user to identify the location of a cancer by selectively exciting the endogenous protoporphyrin IX fluorescent material that contains information on cancer.

As described above, in order to irradiate light from mercury lamp that irradiates light with a short wavelength and from the semiconductor laser that irradiates light with a long wavelength to a skin tissue that is subject to measurement, it will be convenient to deliver lights irradiated from the two different light sources using the same light-guide.

FIGS. 16 and 17 illustrate a typical light source apparatus irradiating light from two different light sources through the same light-guide.

First, FIG. 16 shows a light source apparatus that delivers light to the same light-guide using a dichroic mirror 150. The dichroic mirror 150 is disposed between the optical paths of two light sources, the laser and the lamp, such that lights irradiated from each light source are delivered to the light-guide.

More specifically, as described in FIG. 16, light from the lamp 110 passes through a filter, and then light with a penetration wavelength range of the dichroic mirror selectively passes through the dichroic mirror and is transmitted to the light-guide 130. Also, a laser 120, another light source in FIG. 16, is a light source with wavelength range that is reflected by the dichroic mirror 150, and light from the laser 120 is reflected by the dichroic mirror 150 to be incident to the light-guide 130.

The light source apparatus of such structure relies on the dichroic mirror 150 that separates lights irradiated from the two light sources by their wavelengths and then guides them to the light-guide 130. However, since the dichroic mirror 150 is disposed in the optical path of the lamp light source, an optical loss of light irradiated by the lamp 110 occurs. Particularly, when considering the mercury lamp used under a white light condition, in order for light with a visible light wavelength range irradiated by the mercury lamp to be incident to the light-guide, there is a limitation in that the dichroic mirror needs to be removed from the optical path.

Also, in the light source apparatus with the structure mentioned in the above, there is a limitation in that the filter 140 for the lamp must be provided separately from the dichroic mirror. Also, since the dichroic mirror effectively reflects light only when light is introduced at a specific angle of 45 degrees, the light source design is very limited and the apparatus is difficult to miniaturize.

FIG. 17 illustrates a light source apparatus that delivers lights to the same light-guide by changing the incidence angles of lights from two light sources. In this light source apparatus, a lamp 210 and a laser 220 are disposed to have incidence angles ‘a’ and ‘b’ with respect to the optic axis of light-guide 230, respectively. Thus, lights can be delivered to the same waveguide 230 (unexplained reference numeral 240 indicates a filter).

However, when such optical design in which the incidence angles change is adopted, the incidence angles a and b of two light sources incident to the light-guide 230 has to be set to be large values, reducing the light transmission effect of the light-guide 230.

Meanwhile, in these typical light source apparatus, white light is achieved by combining lamps. In this case, only light of a visible light range is transmitted to achieve white light. Thus, all wavelengths of the visible light range can be achieved. However, although the lamps are combined, it is difficult to achieve optical white light due to a difference between the intensities of lights with the respective wavelength ranges and a recognition difference in Charge-Coupled Device (CCD) sensor.

Also, in case of the lamp light source, since the characteristics of lamp changes according to the lapse of time, the reproducibility of white light is reduced.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention provides a light source apparatus for photo-diagnosis and phototherapy, which can transmit light by combining a plurality of light sources and inhibit harmful spectrum components while increasing the light quantity, extending the optical spectrum, and increasing the uniformity of the illumination spectrum.

The present invention also provides a light source apparatus for photo-diagnosis and phototherapy, which can correct the change of the color temperature of a light source due to the lapse of time to continuously achieve optimal white light.

In one aspect, the present invention provides a light source apparatus for photo-diagnosis and phototherapy, including: a first light source that is non-coherent; a second light source that is coherent; a light-guide delivering light emitted from the first light source and the second light source; an interference filter disposed on an optical path of the first light source; and a compensation filter between the first light source and the light-guide, wherein the light emitted from the second light source is reflected by the interference filter to be incident to the light-guide and the light from the first light source passes through the interference filter at the same time.

In an exemplary embodiment, the interference filter may include a transmission spectrum that transmits main light emitted from the first light source.

In another exemplary embodiment, the second light source may emit light with a wavelength range deviating from a range of the transmission spectrum of the interference filter.

In still another exemplary embodiment, the interference filter may be inclined at a certain angle α with respect to a plane perpendicular to an optical axis of the light-guide.

In yet another exemplary embodiment, the first light source may be inclined at a certain angle α with respect to the optical axis of the light-guide.

In still yet another exemplary embodiment, the certain angle α may range from about 3 degrees to about 10 degrees.

In a further exemplary embodiment, the first light source may include a mercury lamp emitting main emission light in the ultraviolet and visible regions of the spectrum.

In another further exemplary embodiment, the second light source may include a laser emitting a short wavelength light of 500 nm or more.

In still another further exemplary embodiment, the interference filter may have a transmission spectrum with respect to a wavelength range of about 350 nm to about 450 nm.

In yet another further exemplary embodiment, the first light source and the second light source may be disposed such that an incident range of light incident to an incident plane of the light-guide falls within an acceptance angle range of the light-guide, and simultaneously, light spots of the first and second light sources fall within a core of the incident plane of the light-guide.

In still yet another further exemplary embodiment, the compensation filter compensating for an output spectrum of the first light source through converting the output spectrum of the first light source into a predetermined reference output spectrum.

In a still further exemplary embodiment, the compensation filter and the interference filter may constitute a filter wheel so as to be selectively located between the first light source and the light-guide.

In a yet still further exemplary embodiment, the light source apparatus may include an attenuator disposed between the first light source and the filter wheel to control a quantity of light.

In a yet still further exemplary embodiment, the light source apparatus may include a variable diaphragm between the first light source and the filter wheel.

In a yet still further exemplary embodiment, the variable diaphragm may be a movable diaphragm that moves forward or backward to adjust a distance from the first light source.

In a yet still further exemplary embodiment, the variable diaphragm may be configured to vary in aperture size thereof.

In a yet still further exemplary embodiment, the light source apparatus may further include an RGB sensor for sensing an RGB signal of light that passes the filter wheel.

In a yet still further exemplary embodiment, the light source apparatus may further include a diaphragm controller configured to move the variable diaphragm or control an aperture size of the variable diaphragm according to a comparison result of the RGB signal sensed by the RGB sensor and the reference output spectrum.

In a yet still further exemplary embodiment, the filter wheel may further include one or more auxiliary filters that selectively transmit light emitted from the first light source.

Other aspects and exemplary embodiments of the invention are discussed infra.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 is a view illustrating an exemplary light source apparatus according to an embodiment of the present invention;

FIG. 2 is a view illustrating an incidence angle and an output divergence of a lamp and a laser with respect to a light-guide;

FIG. 3 is a view illustrating transmission and reflection spectrums of an interference filter included in a light source apparatus according to an embodiment of the present invention;

FIG. 4 is a view illustrating an exemplary light source apparatus for photo-diagnosis and phototherapy according to an embodiment of the present invention, which can achieve white light in real-time;

FIG. 5 is a view illustrating the characteristics of the output spectrum of a lamp used to achieve white light in a light source apparatus according to an embodiment of the present invention;

FIG. 6 is a view illustrating an exemplary reference output spectrum of white light using a light source apparatus according to an embodiment of the present invention;

FIG. 7 is a view illustrating a design value of a compensation filter;

FIG. 8 is a view illustrating the output characteristics of a compensation filter designed in FIG. 7;

FIG. 9 is a view illustrating a comparison between an output value converted by a compensation filter with such output characteristics and an intrinsic output of a lamp;

FIG. 10 is a view illustrating a change of the output spectrum of an arc lamp according to the lapse of time;

FIG. 11 is a view illustrating a diaphragm disposed at the front of a lamp to compare spectrums at the central and edge parts based on the optical axis of a mercury lamp;

FIG. 12 is a view illustrating spectrums at the central and edge parts based on the optical axis of a mercury lamp;

FIG. 13 is a view illustrating a diaphragm disposed on an optical path of an arc lamp;

FIG. 14 is a graph illustrating a change of an output spectrum of a first light source according to the location change of a variable diaphragm;

FIG. 15 is a view illustrating an exemplary light source apparatus for photo-diagnosis and phototherapy according to an embodiment of the present invention; and

FIGS. 16 and 17 are views illustrating typical light source apparatuses irradiating light from two different light sources through the same light-guide.

Reference numerals set forth in the Drawings includes reference to the following elements as further discussed below:

| | |
|---|---|
| 10: first light source | 20: second light source |
| 30: light-guide | 40: interference filter |
| 50: compensation filter | 60: variable diaphragm |
| 70: attenuator | 80: guide |
| 90: RGB sensor | 100: diaphragm controller |

It should be understood that the accompanying drawings are not necessarily to scale, presenting a somewhat simplified representation of various exemplary features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The above and other features of the invention are discussed infra.

The present invention provides a light source apparatus that is configured to effectively transmit light through a single light-guide from a light source to diagnose and treat various diseases, for example, tumors occurring in the inner and outer parts of the body.

The present invention also provides a light source apparatus that can continuously output white light with an optimal output spectrum.

Hereinafter, a light source apparatus for photo-diagnosis and phototherapy according to an embodiment of the present invention will be described in detail with reference to the accompanying drawings.

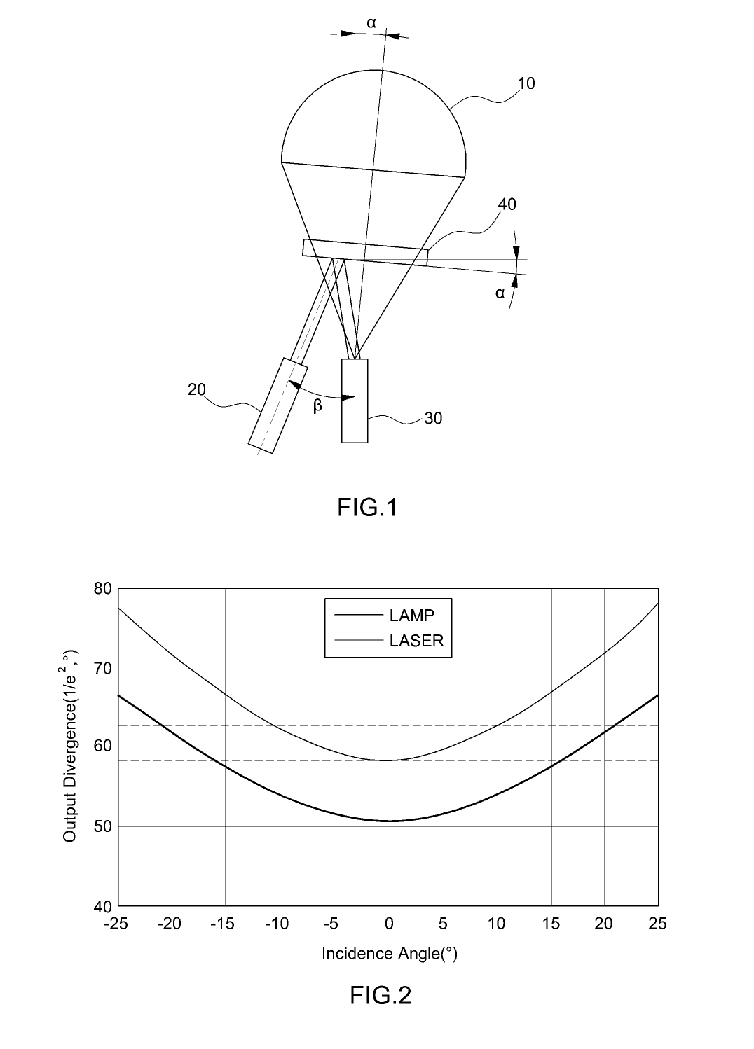

FIG. 1 is a view illustrating an exemplary light source apparatus according to an embodiment of the present invention, in which two light sources are configured to irradiate light through a single light-guide 30.

As shown in FIG. 1, the light source apparatus for photo-diagnosis and phototherapy may include a first light source 10 for emitting non-coherent light and a second light source 20 for emitting coherent light.

The first light source 10 may be a non-coherent light source that irradiates white light on the whole treatment and diagnosis part and has an optical spectral range for excitation. The second light source 20 may be a coherent light source that has a coherent wavelength spectral range for excitation at a specific part of a disease.

The first light source 10 may include a mercury lamp that mainly irradiates light with a wavelength of about 350 nm to about 450 mm. The lamp may be appropriately selected according to factors such as diagnosis and treatment purposes and environments. Also, the second light source 20 may be a long wavelength light source such as laser.

Light emitted from the first light source 10 and the second light source 20 may be configured to be incident to the same light-guide. In an exemplary embodiment, as shown in FIG. 1, the light source apparatus may include a light-guide 30 for transmitting light emitted from the first light source 10 and the second light source 20.

The light-guide 30 may be disposed on the optical path of the first light source 10 to allow light emitted from the first light source 10 to be incident to the light-guide 30.

In an exemplary embodiment, as shown in FIG. 1, the light source apparatus may be configured to include an interference filter 40 with selective transmission and reflection characteristics. The interference filter 40 may be disposed at a location where the optical path of the first light source 10 and the optical path of the second light source 20 overlap each other.

More specifically, the interference filter 40 may be a filter that has a selective transmission characteristic with respect to a specific wavelength range and a high reflection characteristic with respect to other wavelength ranges.

In this embodiment, light emitted from the first light source 10 and the second light source 20 may be configured to be effectively incident to the same light-guide 30 using the characteristics of the interference filter 40. That is, the wavelength range of the main emission light of the first light source 10 and the wavelength range of the main emission light of the second light source 20 may be separated from each other to simultaneously use the transmission and reflection characteristics of the interference filter 40.

For example, the interference filter 40 may be designed to have a transmission spectrum that allows main emission light from the first light source 10 to be transmitted. Thus, irradiation light from the first light source 10 may be mostly transmitted to the light-guide 30.

Accordingly, light emitted from the first light source 10 may pass through the interference filter 40 disposed on the optical path of the first light source 10. In this case, since the main spectral range of light emitted from the first light source 10 may coincide with the transmission spectrum of the interference filter 40, the main emission light of the first light source 10 may be transmitted through the interference filter 40 to be incident to the light-guide 30.

In this embodiment, the second light source 20 may be configured to irradiate light with a wavelength range deviating from the transmission spectral range of the interference filter 40. Accordingly, light emitted from the second light source 20 may be reflected by the interference filter 40 disposed on the optical path of the second light source 20, and then the reflected light may be incident to the light-guide 30.

In this case, the first light source 10 and the second light source 20 may be configured such that the incident range of light incident to the incident plane of the light-guide 30 falls within the acceptance angle range of the light-guide 30, and may be disposed such that the light spots of the first and second light sources 10 and 20 fall within the core of the incident plane of the light-guide 30.

Accordingly, the first light source 10 and the second light source 20 may be compactly disposed such that light emitted from the first light source 10 and the second light source 20 are all incident to the light-guide 30 within the acceptance angle through the transmission or reflection process.

In order to improve the light transmission efficiency, the present invention provides a light source apparatus with a structure that can reduce the incidence angles of the respective light sources.

In an exemplary embodiment, as shown in FIG. 1, the interference filter 40 may be inclined at an inclination angle α with respect to the plane perpendicular to the optical axis of the light-guide. Similarly to the inclination angle α of the interference filter 40, the first light source 10 may also be tilted at the same angle as the inclination angle α such that the coupling angle of the first light source 10 with the respect to the optical axis of the light-guide 30 is identical to the inclination angle α.

Regarding the inclination angle α of the first light source 10, the light-guide 30 may have a numerical aperture that is the maximum acceptance angle at which light can be accepted. When light is incident to the light-guide 30 at an angle larger than the numeral aperture, an optical loss may occur.

FIG. 2 illustrates the incidence angle and the output divergence with respect to the light-guide in a lamp and a laser, respectively. Regarding optical energy with a large incidence angle, since the output divergence increases as much as the incidence angle increases at the end of the light-guide 30, when considering the efficiency, there may be a need for configuration with a small incidence angle.

Accordingly, in an exemplary embodiment of the present invention, the inclination angle α may be set to range from about 3 degrees to about 10 degrees. In this case, as shown in FIG. 2, the output divergence at the end of the light-guide 30 may be controlled below about 62 degrees. When the inclination angle α is set equal to or smaller than about 3 degrees, the second light source 20 disposed at the side of the light-guide 30 may not be mechanically installed at the light-guide 30 and the interference filter 40 due to limitations in size and space, or an optical energy transmission loss may occur.

Meanwhile, light emitted from the second light source 20 in FIG. 1 may be reflected by the interference filter 40 inclined at the inclination angle α, and then may be incident to the light-guide 30. The incidence angle β of the second light source 20 with respect to the optical axis of the light-guide 30 may be set in consideration of the incidence angle of light reflected by the interference filter 40 with respect to the light-guide 30 such that light irradiated from the second light source 20 can be incident to the light-guide 30 within the acceptance angle range thereof.

In this case, the optical energy transmission efficiency of the first light source 10 and the second light source and the similarity of the output divergences of two optical energies at the end of the light-guide 30 need to be considered.

That is, the reflection condition may be set as indicated as a red region of FIG. 2 such that the output divergences of two optical energies at the end of the light-guide 30 are equal to each other and the light sources 10 and 20 have an incident angle smaller than the maximum acceptance angle.

Accordingly, as shown in FIG. 2, when the incident angle of the second light source 20 that is a laser may be set from about 16 degrees to about 22 degrees, the light transmission efficiency and the output divergence at the end of the light-guide 30 can be equally maintained.

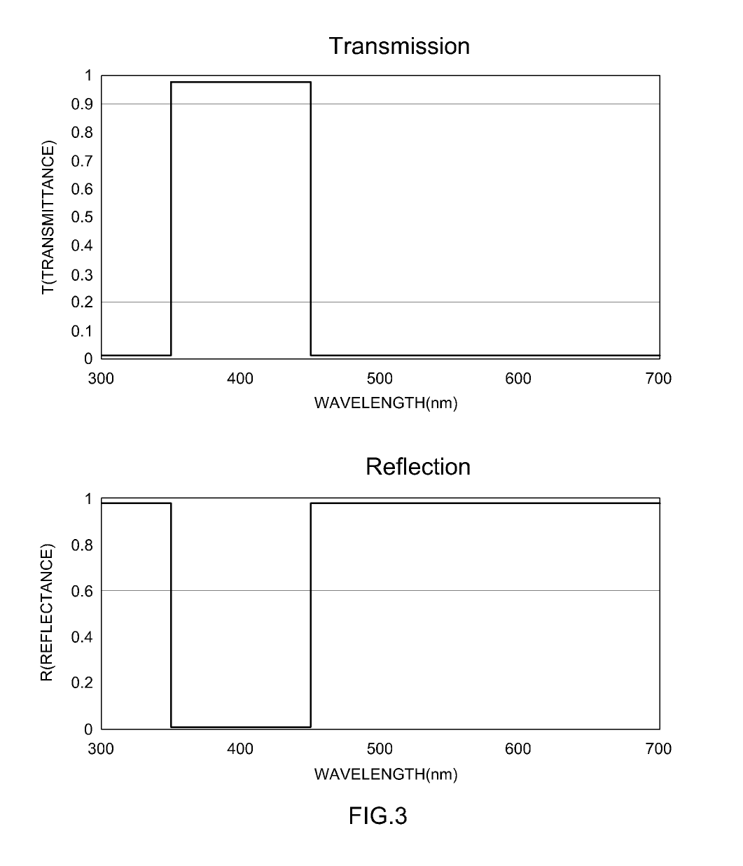

FIG. 3 is a view illustrating the transmission and reflection spectrums of the interference filter 40 designed according to an embodiment of the present invention.

The interference filter 40 may be configured to have a selective penetrating power with respect to a specific wavelength range. In this embodiment, as shown in FIG. 3, the interference filter 40 may be configured to transmit light with a wavelength of about 350 nm to about 450 nm. Meanwhile, the interference filter 40 may reflect light of other wavelengths. i.e., a wavelength equal to or smaller than about 350 nm or equal to or greater than about 450 nm.

The interference filter 40 having the transmission and reflection spectrum as shown in FIG. 3 may be used together with the first light source 10 and the second light source 20 that use the transmission and reflection characteristics.

In case of the interference filter 40, a mercury lamp having main emission light of about 350 nm to about 450 nm may be together used as the first light source 10, and a laser emitting a long wavelength light of about 500 nm or more may be used as the second light source 20. For example, a laser emitting a light of about 635 nm or 660 nm may be together used as the second light source 20.

Here, the first light source 10 and the second light source 20 are not limited to the example as described above. The first light source 10 may be configured such that a part or all selected from ultraviolet or visible region of the spectrum is used as main emission light.

In this case, the interference filter 40 may be configured to selectively transmit light according to the design values of the first light source 10 and the second light source 20.

Accordingly, the light source apparatus for photo-diagnosis and phototherapy can perform effective light transmission by selectively transmitting light from a portion of a plurality of light sources but reflecting light from other portions without an additional optical component such as a dichroic mirror.

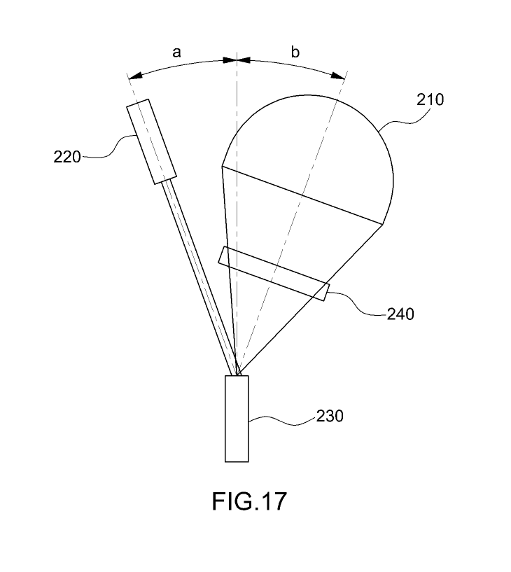

Compared to a typical apparatus shown in FIG. 17, the light source apparatus may be designed such that a difference between incidence angles of light incident to the light-guide 30 is not significant. Particularly, the incidence angle of the second light source 20 may be relatively reduced by the interference filter 40, allowing incidence to the light-guide 30.

Accordingly, the first and second light sources 10 and 20 may be disposed such that the incidence ranges of the first light source 10 and the second light source 20 fall within the acceptance angle range of the light-guide 30, and simultaneously, the light spots of the light sources 10 and 20 may fall within the core of the incident plane of the light-guide 30.

In the light source apparatus for photo-diagnosis and phototherapy, the irradiation use efficiency of the light source can be increased, and the structure of the light source apparatus can be simplified by using the same interference filter 40 on the optical path of the second light source 20 such as a laser as well as the optical path of the first light source 10 such as a lamp.

The light source apparatus for photo-diagnosis and phototherapy may be configured to achieve a white light mode for providing white light to observe diagnosis and treatment parts in the photo-diagnosis and phototherapy processes.

In the white light mode, the first light source 10 that is non-coherent may be used, and a filter and an attenuator 70 may be used to acquire an output close to white light.

Particularly, the output of the light source in white light mode may be processed and maintained closest to white light during the entire usage time.

For this, the light source apparatus may further include a variable diaphragm 60 and a compensation filter 50 between the first light source 10 and the light-guide 30.

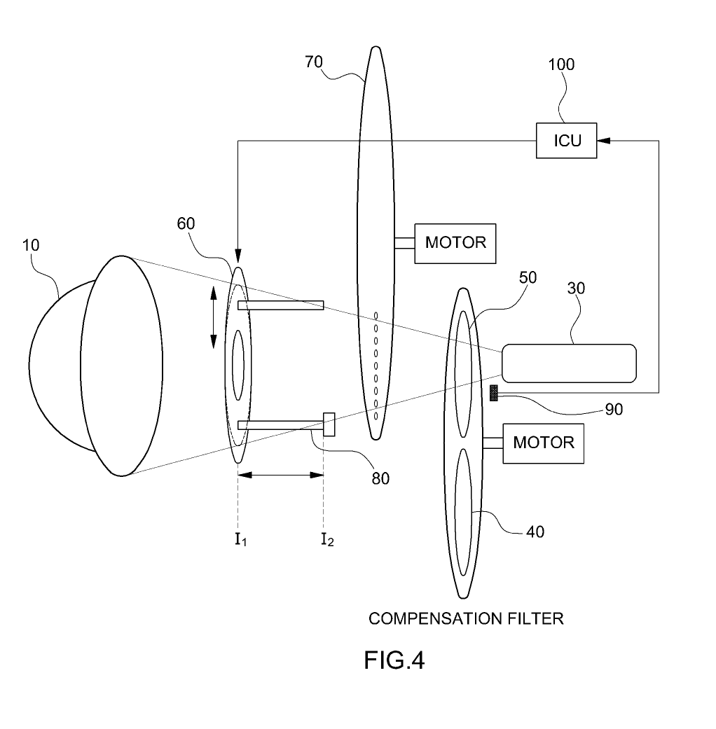

In this regard, FIG. 4 illustrates an exemplary light source apparatus for photo-diagnosis and phototherapy according to an embodiment of the present invention, which can achieve white light in real-time.

As shown in FIG. 4, the variable diaphragm 60 and the compensation filter 50 may be disposed on the optical path from the first light source 10 to the light-guide 30.

The compensation filter 50 may convert light emitted from the first light source 10 into a form of white light having a desired output spectrum. The compensation filter 50 may be a white light conversion filter that is configured to selectively absorb or transmit light of a specific wavelength range.

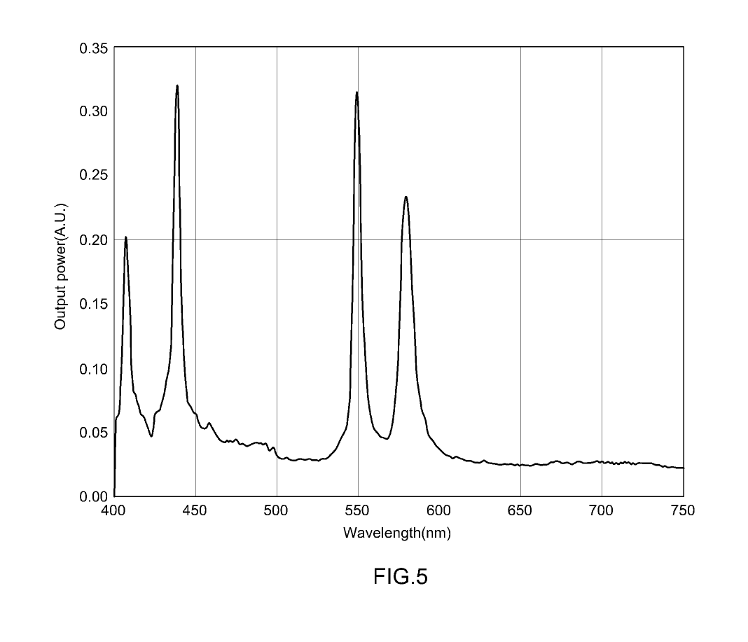

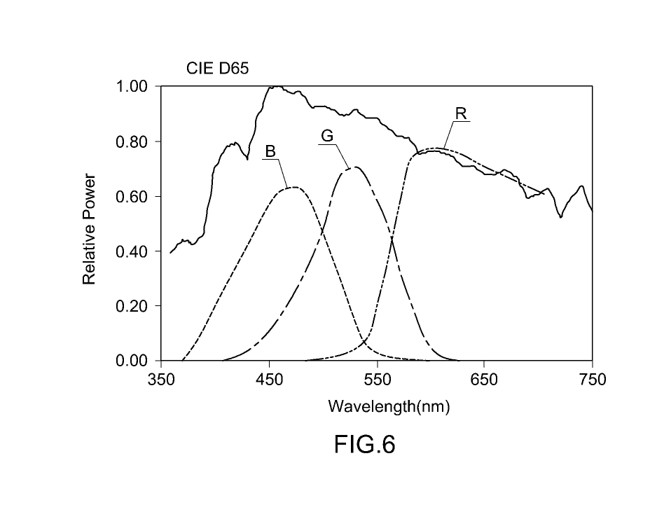

In this regard, FIG. 5 illustrates an output spectrum in a visible light range of a mercury lamp used as the first light source 10. FIG. 6 illustrates a reference output spectrum for white light.

Referring to FIGS. 5 and 6, since a white light source shows a great difference from the reference output spectrum, there are difficulties in implementing optimal white light.

In order to overcome these limitations, the compensation filter 50 may be disposed on the optical path to convert lamp light of an output spectrum as shown in FIG. 5 into a reference output spectrum of FIG. 6.

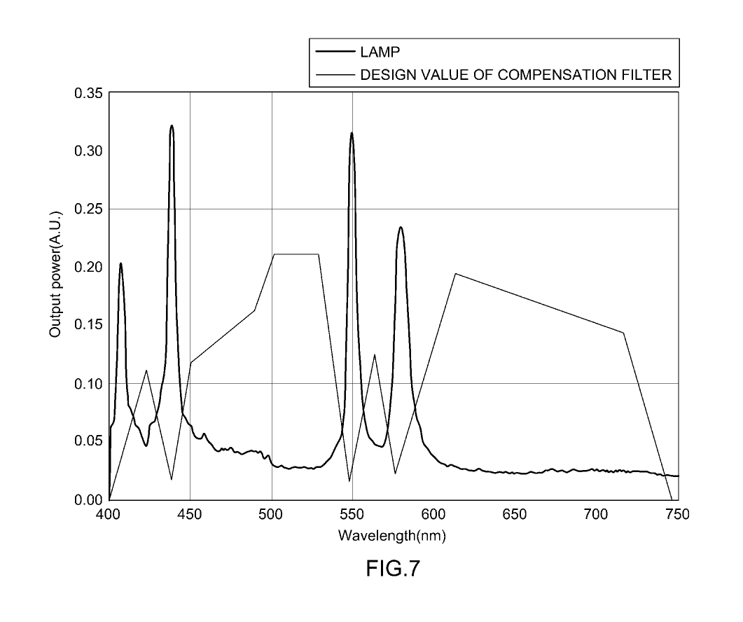

FIG. 7 illustrates a design value of the compensation filter 50 according to the sensitivity of the RGB (Red, Green and Blue) range of a CCD sensor, which is implemented to have transmittance and slope at a specific wavelength range.

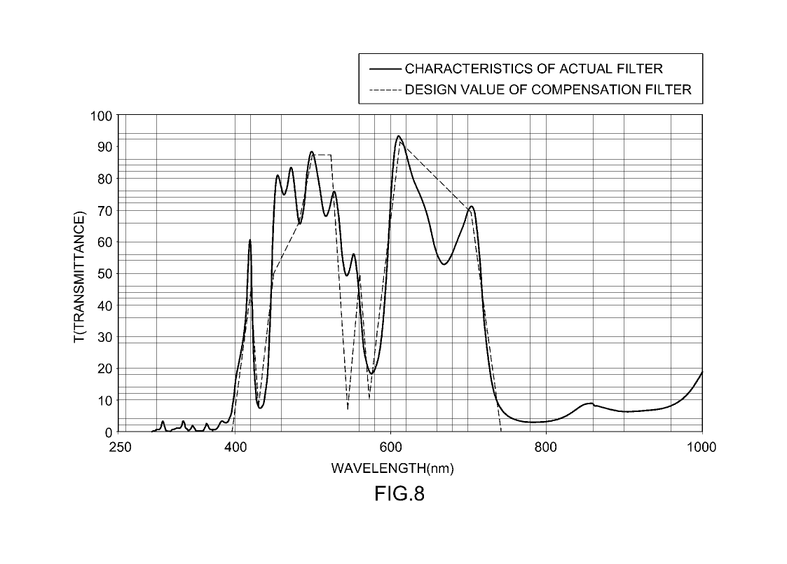

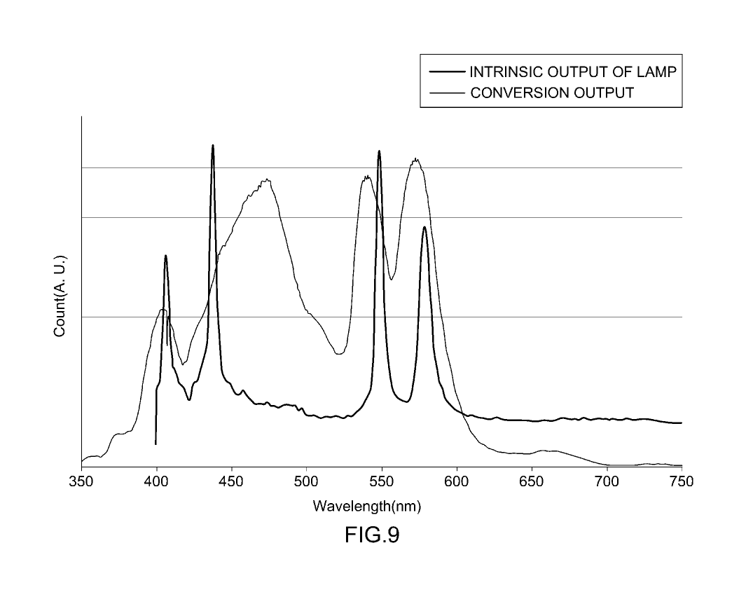

FIG. 8 shows the transmission characteristics of the compensation filter 50 that is actually designed based on the design value. It can be seen that the actual filter characteristics are similar to the design value of the compensation filter 50. FIG. 9 shows output values converted using the compensation filter 50 having such transmission characteristics. It can be seen that the conversion output spectrum by compensation is similar to the reference output spectrum compared to the intrinsic output of the lamp.

Accordingly, the light source apparatus for photo-diagnosis and phototherapy may include the compensation filter 50 between the first filter 10 and the light-guide 30, and thus may provide a high quality of white light by converting the output spectrum of the first light source 10 into a predetermined reference output spectrum using the compensation filter 50.

The compensation filter 50 and the interference filter 40 described above may be selectively used. For example, the interference filer 40 and the compensation filter 50 may be manufactured in a form of filter wheel. The filter wheel including the interference filter 40 and the compensation filter 50 may be rotated by a motor connected thereto, and may be located on the optical path. Accordingly, white light, excited light, or mixed light may be selectively provided according to the need in the process of photo-diagnosis and phototherapy.

The filter wheel may be configured to include one or more auxiliary filters that selectively transmit light irradiated from the first light source 10. The auxiliary filter may transmit only light of a specific wavelength range through the light-guide 30 according to the need.

Furthermore, the light source apparatus for photo-diagnosis and phototherapy may further include an attenuator 70 disposed between the first light source 10 and the filter wheel to control the quantity of light. The attenuator 70 may be configured to be rotatable by a motor like the interference filter 40 and the compensation filter 50 so as to adjust the degree of attenuation.

Typical lamps used as the white light source may show a change of the output spectrum according to the lapse of time. In an exemplary embodiment, the light source apparatus may include a variable diaphragm 60 for correcting the change of the output spectrum.

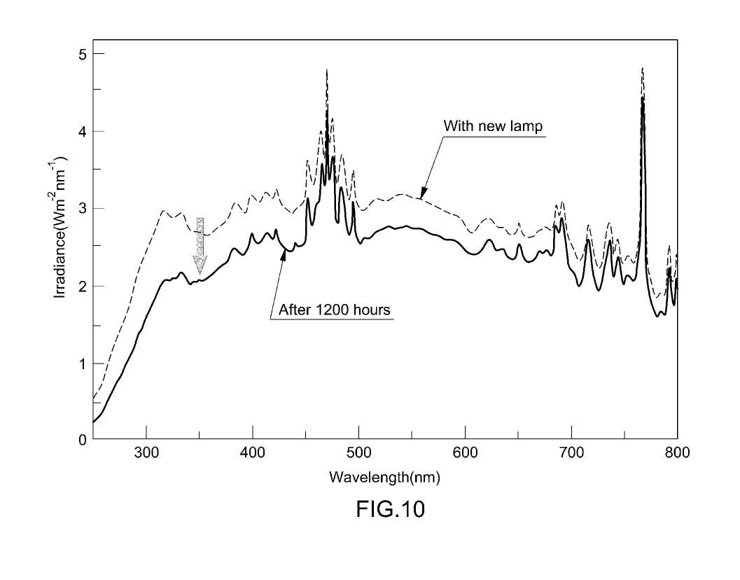

In this regard, FIG. 10 shows the change of the output spectrum of the lamp, i.e., the change of the color temperature according to the lapse of time. Referring to FIG. 10, when an arc lamp is used for about 1,200 hours, it can be seen that the arc lamp becomes relatively remarkable in red class compared to a new lamp.

Accordingly, due to the change of the color temperature of the light source as shown in FIG. 10, the light source apparatus as shown in FIG. 4 shows the same output value as the reference output spectrum originally designed only for a certain time at the initial stage, and then shows a change output value after the lapse of certain time. Accordingly, when only the compensation filter 50 is simply applied, it may become difficult to continuously achieve optimal white light.

Meanwhile, the present applicant confirmed that the intensity of an RGB signal shows a certain tendency, by studying the characteristics of the color temperature according to the output divergence of the mercury lamp. The blue and green regions were dominantly shown at the outer side of the optical path from the mercury lamp.

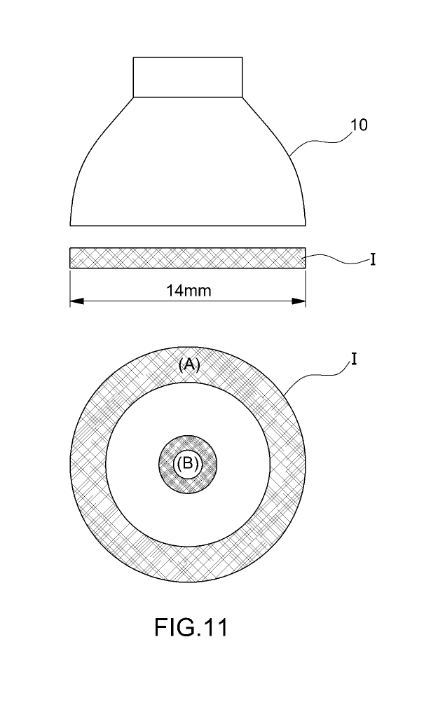

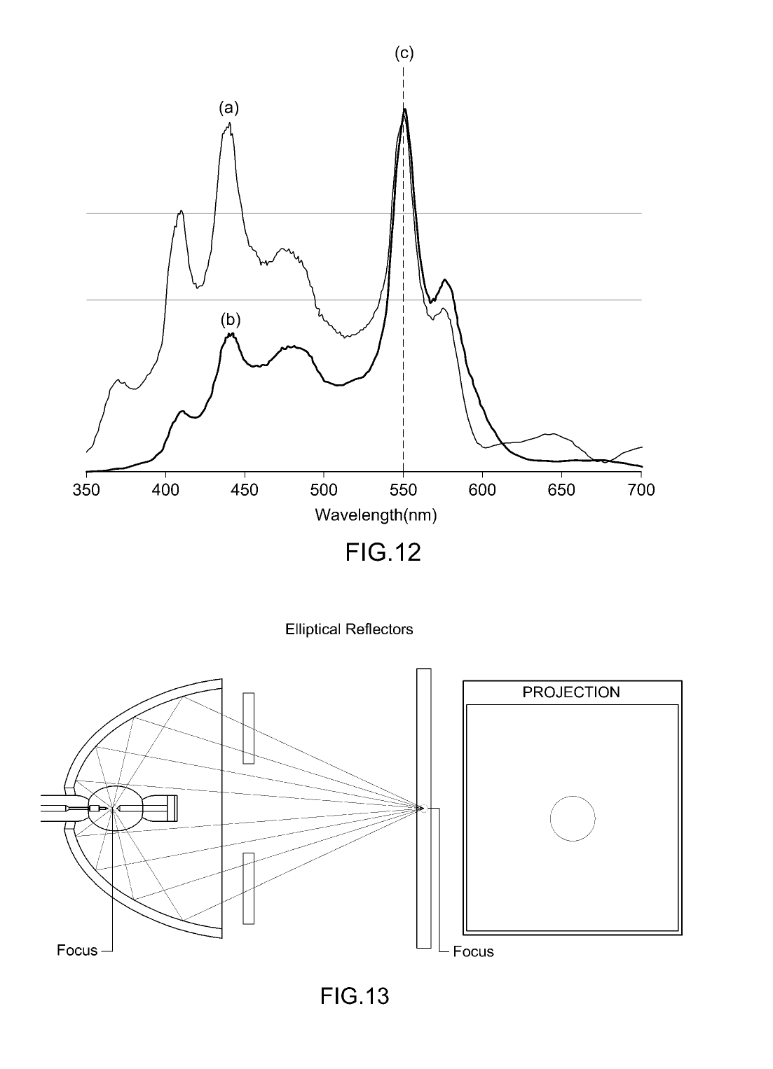

FIGS. 11 and 12 illustrate spectrums at the central and edge parts based on the optical axis of a mercury lamp.

As shown in FIG. 11, a diaphragm I was installed at the front end of the mercury lamp, and the output spectrum of the lamp was measured at the edge part A and the central part B. The measurement results are shown in FIG. 12.

Particularly, two data were normalized based on a wavelength C of 550 nm to compare and analyze the spectral characteristics. From this, it can be seen that the graph A with respect to the edge part is dominant in the blue and green regions compared to the graph B with respect to the central part.

Accordingly, the light source apparatus for photo-diagnosis and phototherapy may include the variable diaphragm 60 between the first light source 10 and the filter wheel to control the output spectrum of optical energy transmitted to the light-guide 30 by selectively interrupting light with respect to the edge part of the first light source.

Accordingly, the light source apparatus can actively control the change of the intensity of the RGB signal compared to the output spectrum that is originally, caused by the change of the output of the light source according to the lapse of time.

As shown in FIG. 13, the variable diaphragm 60 may be disposed to block light outwardly irradiated from the optical path of the arc lamp. The intensity of the red region may be corrected so as not to increase according to the lapse of time by controlling the blocking range of light outwardly irradiated. That is, in order to correct the red region that increases in its intensity according to the lapse of time, the variable diaphragm 60 may be allowed to less block the outer region of the lamp to compensate for the blue and green regions.

Accordingly, the variable diaphragm 60 may selectively block a portion of light irradiated from the first light source 10 and incident to the light-guide 30 from the outer side based on the optical axis to correct the RGB balance. Thus, the condition similar to the initial reference output spectrum may be maintained.

The variable diaphragm 60 may be implemented in a type in which the size of the aperture is adjusted or a type in which the variable diaphragm 60 can move forward or backward along a guide 80 disposed on the optical path.

That is, the variable diaphragm 60 may be configured to move forward or backward on the optical path or change in its aperture size to set the blocking region of the lamp.

For example, when light dominant in red region is irradiated according to the lapse of time, and as shown in FIG. 4, the variable diaphragm 60 moves from a location I1 where the variable diaphragm 60 is originally disposed to a location I2 closer to the light-guide 30, the intensity of the wavelength range of the blue and green classes may increase, thereby compensating for the increase of the intensity of the wavelength range of the red classes.

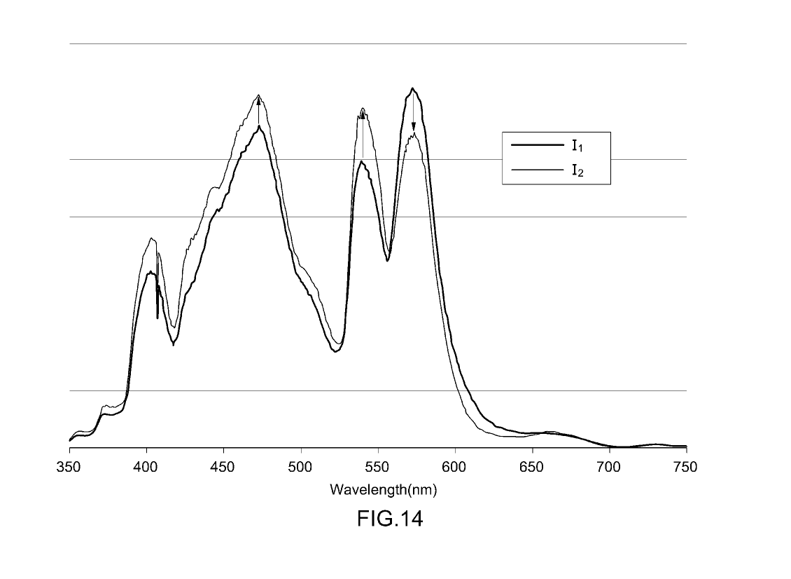

FIG. 14 is a graph illustrating the change of the output spectrum of the first light source 10 according to the location change of the variable diaphragm 60, which shows a comparison between the output spectrums of the lamp at the location I1 where the variable diaphragm 60 is originally disposed and the location I2 closer to the light-guide 30.

Referring to FIG. 14, as the variable diaphragm 60 moves from the location I1 where the variable diaphragm 60 is originally disposed to the location I2 closer to the light-guide 30, the blocking degree of the outer region of the variable diaphragm 60 can be reduced, showing the effect in which the intensity of the wavelength range of the blue and green classes is strengthened. Accordingly, since the effect in which the intensity of the wavelength range of the red class is relatively strengthened due to the lifespan of the lamp can be offset, the output condition of white light that is originally set can be maintained.

In the structure in which the aperture size of the diaphragm 60 is adjustable, when the intensity of the wavelength range of red class may be strengthened according to the lapse of time, and the aperture size of the diaphragm can be widened, the degree of blocking the outer region of the lamp can be reduced. Thus, the same effect as the variable diaphragm 60 moves can be achieved.

In order to perform the above process, the variable diaphragm 60 may be configured to further include a diaphragm controller to control the movement and aperture size of the variable diaphragm 60

The diaphragm controller may check light incident to the light-guide 30, and then may move forward and backward the variable diaphragm 60 or change the aperture size of the variable diaphragm 60.

For this, the light source apparatus may be configured to include an RGB sensor 90 to detect an RGB signal of light that passes the filter wheel

FIG. 4 illustrates a light source apparatus for photo-diagnosis and phototherapy including the diaphragm controller 100 and the RGB sensor 90. As shown in FIG. 4, the RGB signal may be acquired in real-time by the RGB sensor 90. The RGB signal may be delivered to the diaphragm controller 100. According to the comparison result of the reference spectrum data of initial white light, the diaphragm controller 100 may produce white light in real-time by controlling the aperture size or the location of the variable diaphragm 60.

Unlike FIG. 4, optimal white light can be induced in real-time by automatically or manually controlling the variable diaphragm 60 through a CCD sensor, a photodiode having a filter, a spectrometer, or a naked eye.

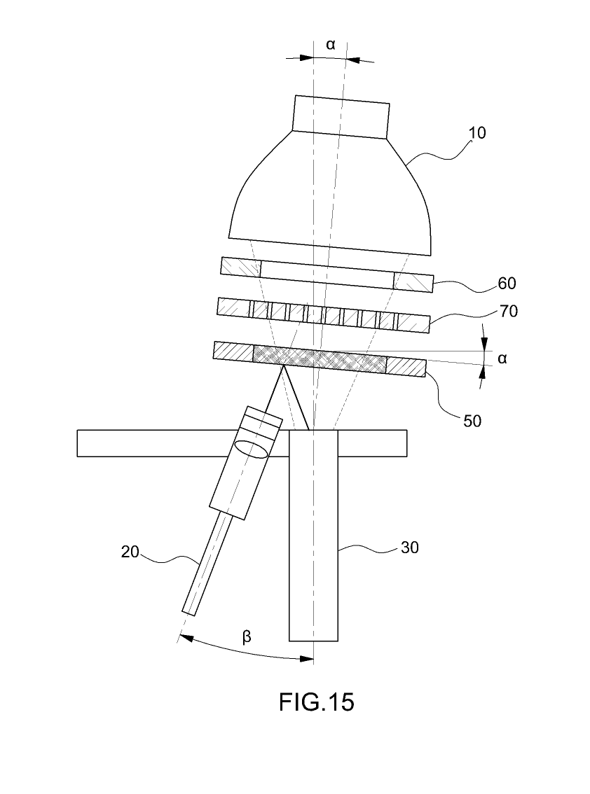

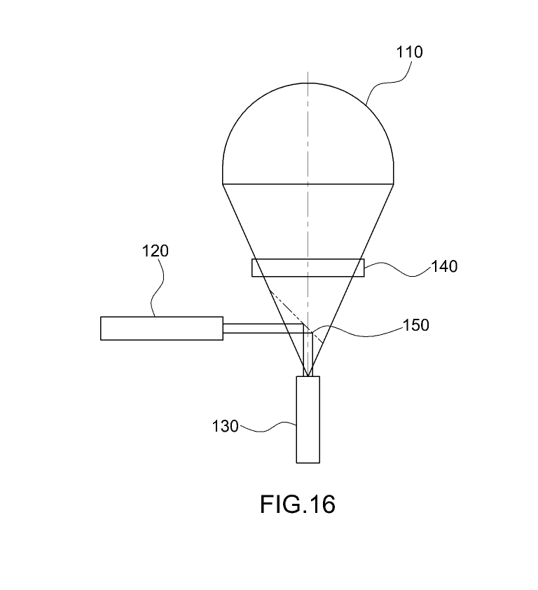

FIG. 15 is a view illustrating an exemplary light source apparatus including a coherent second light source 20 according to an embodiment of the present invention. However, the configuration except the attenuator 70, the variable diaphragm 60, and the compensation filter 50 is similar to that of FIG. 1.

As described above, the compensation filter 50 may be located instead of the interference filter 40, and the attenuator 70 and the variable diaphragm 60 may be disposed between the compensation filter 50 and the first light source 10.

In this case, when the interference filter 40 is inclined at an angle α, the compensation filter 50 for replacing the interference filter 40 may be inclined at the same inclination angle. The attenuator 70 and the variable diaphragm 60 may also be inclined at the same angle as the inclination angle of the interference filter 40.

As described above, a light source apparatus for photo-diagnosis and phototherapy according to an embodiment of the present invention has the following effects.

First, since the incident angle to a light-guide with respect to light irradiated from light sources can be reduced, the light source apparatus can reduce an optical loss at the light-guide, thereby increasing the quantity of light.

Second, the light source apparatus selectively can transmit only a wavelength range of visible light and achieve optimal white light using a compensation filter.

Third, the light source apparatus can continuously achieve optimal white light until the replacement of a lamp by controlling the change of the color temperature according to the lifespan of the lamp.

The invention has been described in detail with reference to exemplary embodiments thereof. However, it will be appreciated by those skilled in the art that changes may be made in these embodiments without departing from the principles and spirit of the invention, the scope of which is defined in the appended claims and their equivalents.

1. A light source apparatus for photo-diagnosis and phototherapy, comprising:
a first light source that is non-coherent;
a second light source that is coherent;
a light-guide delivering light emitted from the first light source and the second light source;
an interference filter disposed on an optical path of the first light source; and
wherein the light emitted from the second light source is reflected by the interference filter to be incident to the light-guide, and the light from the first light source passes through the interference filter at the same time.

2. The light source apparatus of claim 1, wherein the interference filter comprises a transmission spectrum that transmits main light emitted from the first light source.

3. The light source apparatus of claim 2, wherein the second light source emits light with a wavelength range deviating from a range of the transmission spectrum of the interference filter.

4. The light source apparatus of claim 1, wherein the interference filter is inclined at a certain angle with respect to a plane perpendicular to an optical axis of the light-guide.

5. The light source apparatus of claim 4, wherein the first light source is inclined at a certain angle with respect to the optical axis of the light-guide.

6. The light source apparatus of claim 4, wherein the certain angle ranges from about 3 degrees to about 10 degrees.

7. The light source apparatus of claim 1, wherein the first light source comprises a mercury lamp emitting main emission light in the ultraviolet and visible regions of the spectrum.

8. The light source apparatus of claim 7, wherein the second light source comprises a laser emitting a long wavelength light of 500 nm or more.

9. The light source apparatus of claim 7, wherein the interference filter has a transmission spectrum with respect to a wavelength range of about 350 nm to about 450 nm.

10. The light source apparatus of claim 1, wherein the first light source and the second light source are disposed such that an incident range of light incident to an incident plane of the light-guide falls within an acceptance angle range of the light-guide, and simultaneously, light spots of the first and second light sources fall within a core of the incident plane of the light-guide.

11. The light source apparatus of claim 1, comprising a compensation filter between the first light source and the light-guide, the compensation filter converting an output spectrum of the first light source into a predetermined reference output spectrum

12. The light source apparatus of claim 11, wherein the compensation filter and the interference filter constitute a filter wheel so as to be selectively located between the first light source and the light-guide.

13. The light source apparatus of claim 12, comprising an attenuator disposed between the first light source and the filter wheel to control a quantity of light.

14. The light source apparatus of claim 11, comprising a variable diaphragm between the first light source and the filter wheel.

15. The light source apparatus of claim 14, wherein the variable diaphragm is a movable diaphragm that moves forward or backward to adjust a distance from the first light source.

16. The light source apparatus of claim 14, wherein the variable diaphragm is configured to vary in aperture size thereof.

17. The light source apparatus of claim 14, further comprising an RGB sensor for sensing an RGB signal of light that passes the filter wheel.

18. The light source apparatus of claim 17, further comprising a diaphragm controller configured to move the variable diaphragm or control an aperture size of the variable diaphragm according to a comparison result of the RGB signal sensed by the RGB sensor and the reference output spectrum.

19. The light source apparatus of claim 12, wherein the filter wheel further comprises one or more auxiliary filters that selectively transmit light emitted from the first light source.
